(19) United States
(12) Patent Application Publication
(10) Pub. No.: US 2006/0271155 A1
(43) Pub. Date: Nov. 30, 2006

(54) Plaque remover

(76) Inventors: Michael Herr, Philadelphia, PA (US); Marek Swaboda, Philadelphia, PA (US); Michael Zagorsky, Philadelphia, PA (US)

Correspondence Address: Michael Herr, 333 Queen St., Philadelphia, PA, 19147, US

(21) Appl. No.: 11/443,782

(22) Filed: May 31, 2006

Related U.S. Application Data
(60) Provisional application No. 60/685,164, filed on May 31, 2005.

(51) Int. Cl. A61F 2/06

(52) U.S. Cl. 623/1.11

(57) ABSTRACT

A method and apparatus for treating occluded or partially occluded vessels comprising a device for treating occluded or partially occluded vessels comprising, a double-balloon inflatable device being deployed by a catheter having a central channel for continuous blood flow, wall of central channel includes a cutting or abrasion instrument for disrupting the epithelium of the vessel wall, said balloons provide a washable volume for introducing plaque dissolving fluid that is pumped in an inlet and out an outlet, and a stent is located in the lumen of the device and can be deployed after the plaque removal process when the devices is being removed.

### CROSS-REFERENCE TO RELATED APPLICATION

This application claims the benefit of U.S. Provisional Application No. 60/685,164, filed May 31, 2005, the entire disclosure of which is incorporated herein by reference.

### FIELD

This invention related to balloon angioplasty devices. More particularly, the present invention is directed non-occlusive, distal protection, balloon angioplasty devices.

### BACKGROUND

Cardiovascular disease is the main cause of death in United States, claiming over 1.4 million lives per year. More than 70 million of Americans suffer from some type of cardiovascular disease, which in 2002 cost them almost $400 billion in direct and indirect costs

An estimated of over 7 million people will suffer from heart attack and almost 500,000 will die this year as a result of heart attack or coronary heart disease; another 167,200 will die from stroke. This translates to estimated total cost of CHD to over $140 billion ($70B direct) and that of stroke to over $50 billion ($35B direct).

Currently the two main procedures performed to treat the accumulation of fat (and other elements) that forms plaques in the arteries are: Coronary Artery Bypass Graft (CABG) surgery and the Percutaneous Transluminal Coronary Angioplasty (PTCA) (commonly referred as angioplasty), which recently is almost exclusively combined with stenting procedure. In most cases, those treatments are successful in regulating the blood flow only temporarily and suffer from many drawbacks. Neither bypass nor angioplasty solves the patients' problems permanently. Angioplasty procedure leaves plaque in patient's artery and allows tissue grow around the stent, which often results in artery's re-occlusion requiring repeated treatment or bypass surgery. The use of new drug eluting stents (DES) alleviates the problem of restenosis, but not completely.

Bypass procedure also does not remove plaques and is used only when the patient does not qualify for angioplasty. The reason is that major open-heart surgery brings on many risks i.e. infection, anesthesia complications, etc. The biggest drawback of CABG is the high cost and time including surgery and recuperation period. One can easily see the need for a technology that is safe, fast, and minimally invasive that ideally removes the arterial plaque effectively without possible damage to rest of the body parts.

Other novel methods to treat atherosclerosis or thrombosis are emerging. Some prototype devices remove plaques for example, using high-energy lasers or intravascular drills or burrs, while others utilize microwaves or ultrasonic waves but they are unsafe, because of a chance of damaging the arterial wall, possibility of plaque rupture and subsequently formation of a blood clot. Most devices occlude the artery during the procedure increasing the risk of medical complications. Various complications including the irreparable damage of the inner artery wall, plaque rupture, and blood clot formation may occur while using the intravascular drills.

Devices such as those disclosed in U.S. Pat. Nos. 5,833,650 and 6,622,732 refer to distal protection devices but they do not provide continuous flow of blood through the blood vessel.

Therefore, there is a need for safer devices, removing arterial plaques, not occluding artery during the procedure, securing the artery against plaque rapture and debris

### SUMMARY

The invention herein disclosed is a novel method and apparatus for treating partially occluded or fully occluded blood vessels. The invention allows removal of plaque from the lumen of blood vessels such as the coronary or carotid arteries. An object of the invention is to remove plaque from the lumen of a blood vessel while also allowing continuous blood flow through the device during the plaque removal process. Another object of the invention is to allow long durations of plaque removal by allowing continuous blood flow. Another object of the invention is to provide a contained working area for injection and removal of fluids to remove plaque while maintaining continuous blood flow through the blood vessel. Another object of the invention is to provide inflatable tubular barriers at the proximal and distal ends of the device and an enclosed work area to protect against the release of embolic debris during the plaque removal process. Another object of the invention is to provide a means for disrupting the endothelium of the blood vessel to expose the plaque for removal. Another object of the invention is to deploy a stent after the plaque removal process so a second stenting procedure is not necessary and can occur in one complete procedure along with the plaque removal process. Another object of the invention is to traverse the blood vessel during the plaque removal process to remove a long tracks of plaque.

### BRIEF DESCRIPTION OF THE DRAWINGS

In the drawings, which are discussed below, one or more preferred embodiments are illustrated, with the same reference numerals referring to the same pieces of the invention throughout the drawings. It is understood that the invention is not limited to the preferred embodiments depicted in the drawings herein, but rather it is defined by the claims appended hereto and equivalent structures.

FIG. 1. Plaque remover general view

FIG. 2. Plaque remover cross-section view.

FIG. 3. Deflated Plaque Remover inserted into the artery

FIG. 4. Inflated Plaque Remover in the artery dissolving plaque

FIG. 5. Rendering of inflated plaque remover

### DETAILED DESCRIPTION

While the specification concludes with claims particularly pointing out and distinctly claiming the subject matter that is regarded as the invention, the invention will now be further described by reference to the following detailed description of preferred embodiments taken in conjunction with the above-described accompanying drawings.

The present invention comprises a device for performing angioplasty wherein a non-occulusive balloon angioplasty device is used to remove plaque, clog, obstruction or accumulation from the artery lumen, pipe, channel etc. without cutting off the flow in the artery, pipe or channel, and without using mechanical force to extend the diameter of the artery, pipe or channel.

Instead, this device separates the plaque or other accumulation inside the lumen, dissolves the plaque, and washes out debris. During the whole procedure the flow in the artery, pipe or channel is continuously maintained.

The device consists of acatheter, catheter extension, and inflatable double-tire-type balloon. The double-tire-type balloon is mounted on the catheter. An embodiment of the device includes a cutting or abrasion instrument on the inner wall of the invention to disrupt the epithelium and expose the plaque. Another embodiment of the invention includes a means to deploy a stent from the lumen of the invention so that after the plaque removal process, a stent can be deployed in the area of plaque removal.

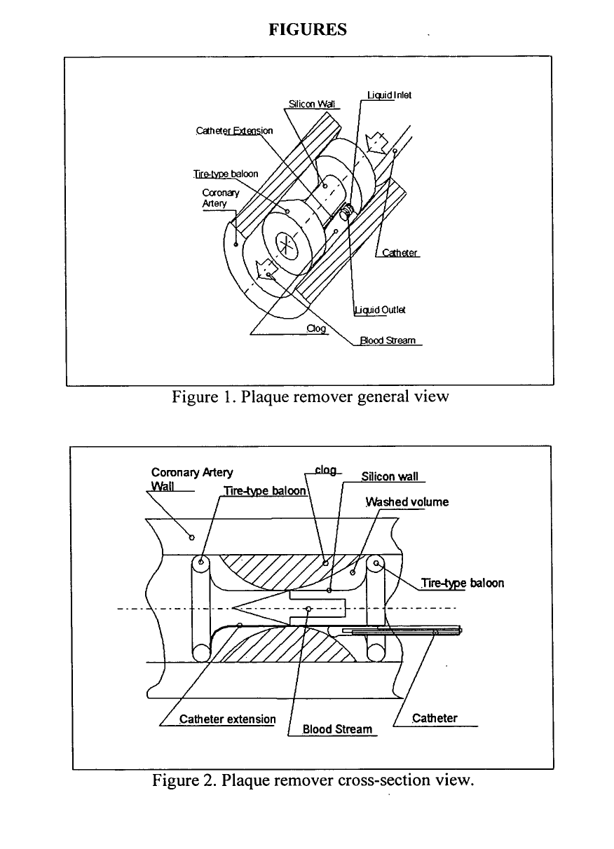

The double-tire-balloon consists of two o-ring shape balloons connected with an elastic sleeve (silicon wall). See FIG. 1. and FIG. 2. The device provides a central channel to allow blood flow to continuously pass through the device.

3-D view in FIG. 1 and cross-section view in FIG. 2 shows how the plaque region is isolated while flow is secured by the Plaque Remover channel placed in the center of the double-tire-type balloon.

Principle of Operation.

An artery is used as an example.

STEP I

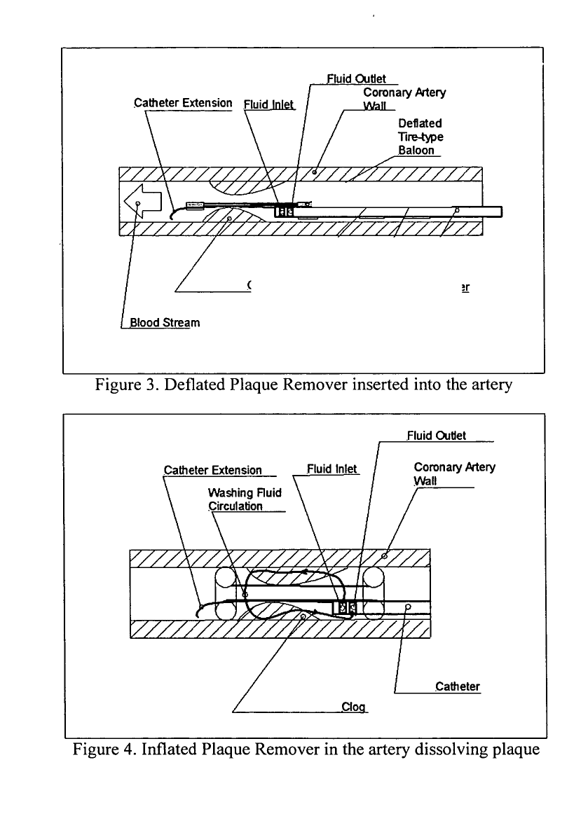

The device is inserted into to the artery, and placed on the plaque or clog as shown in FIG. 3.

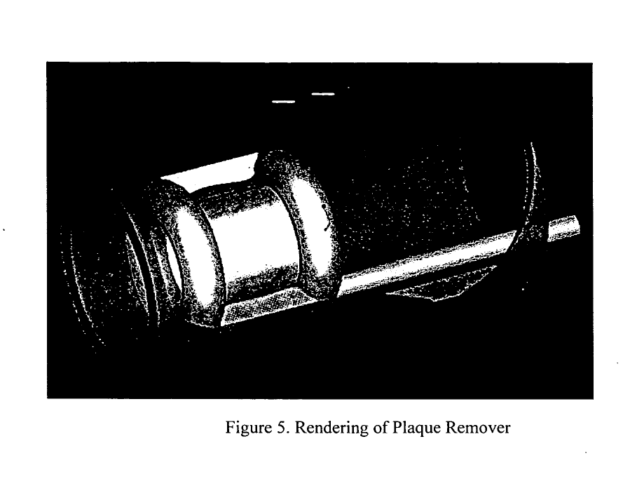

STEP II. The double-tire-type balloon is inflated as shown in FIG. 4. The washable volume is cut off from the blood stream by the double-tire-type balloon, while the blood flow through the device is contionuous.

STEP III. A cutting or abrasion instrument attached to the wall of the device is used to disrupt the epithelium and expose the plaque.

STEP IV. The dissolving liquid pump starts to deliver the clog dissolving solution to the washable volume via inlet port, at the same time the same amount of liquid is evacuated from the washable volume (as depicted in FIG. 4) until clog is dissolved and pumped out.

STEP V. The liquid pump is stopped, double-tire-type balloon is deflated, the device is removed.

STEP VI. Upon removal of the device, a stent is deployed from the lumen of the device in the location where the plaque was removed.

The present invention may be embodied in other specific forms without departing from the spirit or essential attributes thereof and, accordingly, reference should be made to the appended claims, rather than to the foregoing specification, as indicating the scope of the invention.

What is claimed is:

1. A device for treating occluded or partially occluded vessels comprising: a double-balloon inflatable device being deployed by a catheter and having a central channel for continuous blood flow, wall of central channel includes a cutting or abrasion instrument for disrupting the epithelium of the vessel wall, said balloons provide a washable volume for introducing plaque dissolving fluid that is pumped in an inlet and out an outlet, and a stent is located in the lumen of the device and can be deployed after the plaque removal process when the devices is being removed.

2. A method for treating occluded or partially occluded vessels comprising: inserting the device into to the artery, and placing the device on the plaque or clog, inflating the double-tire balloon to create a washable volume that prevents release of debris during the plaque removal process, providing a central channel of the device to allow blood flow to continuously pass through the device, using a cutting or abrasion instrument attached to the wall of the device to disrupt the epithelium and expose the plaque, injecting a plaque dissolving liquid pumped to the washable volume via an inlet port, at the same time the same amount of liquid is evacuated from the washable volume, until clog is dissolved and pumped out, then the liquid pump is stopped and the double-tire-type balloon is deflated, and the device is removed while deploying a stent in the place of plaque removal.
